(19) United States
(12) Patent Application Publication
(10) Pub. No.: US 2026/0363994 A1
(43) Pub. Date: Nov. 19, 2026

(54) GLOSSY, NON-TRANSFER COMPOSITION

(71) Applicant: L V M H RECHERCHE, Saint Jean De Braye (FR)

(72) Inventors: Valérie BOUCHARD DE LA POTERIE, Saint Jean De Braye (FR); Elodie CHAMPREDONDE, Saint Jean De Braye (FR); Maud JAFFRE, Saint Jean De Braye (FR); Anne-Sophie RATTINA, Saint Jean De Braye (FR)

(73) Assignee: L V M H RECHERCHE, Saint Jean De Braye (FR)

(21) Appl. No.: 19/144,257

(22) PCT Filed: Dec. 29, 2023
(86) PCT No.: PCT/FR2023/052138

§ 371 Date: Jun. 27, 2025

(30) Foreign Application Priority Data
Dec. 30, 2022 (FR) FR2214705

(51) Int. Cl. A61K 8/73; A61K 8/34; A61K 8/87; A61K 8/92; A61Q 1/06; A61Q 1/10; A61Q 5/00; A61Q 19/00

(52) U.S. Cl. CPC A61K 8/731; A61K 8/34; A61K 8/87; A61K 8/92; A61Q 1/06; A61Q 1/10; A61Q 5/00; A61Q 19/00

(57) ABSTRACT

The present invention relates in particular to an anhydrous cosmetic composition comprising, in a physiologically acceptable medium:
(i) at least one alkylcellulose, preferably ethylcellulose,
(ii) at least one film-forming polyurethane polymer, and
(iii) at least one non-aqueous polar volatile solvent selected from among C1-C5 lower monoalcohols, volatile hydrocarbon oils selected from within the group consisting of optionally hydroxylated esters, ethers, dialkoxymethane, carbonates, and mixtures thereof.

### FIELD OF THE INVENTION

The present invention relates to the field of cosmetics. It relates in particular to glossy, non-transfer anhydrous compositions for care and/or makeup of keratin materials, in particular of the skin or lips.

### STATE OF THE ARTAS

Known from the prior art are care and/or makeup compositions of keratin materials with glossiness and hold, but which have poor resistance to transfer to pressure or friction, that is to say they tend to be deposited on a support (skin, clothing, etc.) with which they are brought into contact (these products are said to transfer by touch or friction). In the world of lipsticks in particular, an ultra-glossy product (‘vinyl’ appearance) with very long-lasting hold or even no transfer is sought.

However, to the Applicant's knowledge, there are currently no such products on the market which offer very long-lasting hold with non-transfer properties and easy removal.

Gloss products have the characteristic of providing a very glossy or “vinyl” film on the lips, but they have the disadvantage of depositing a film with little coverage and little intense color with often limited hold over time, significant migration in fine lines and significant transfer. Stick products, on the other hand, have the particularity of providing an intense color with a longer hold but are not non-transfer, and have a less significant glossiness than a gloss; sticks said to be long-lasting but not ‘non-transfer’ generally have satin films (not glossy films) and ‘non-transfer’ sticks are matte and are less comfortable, even drying.

Some products claim long-lasting glossiness, but they usually have a sticky feel, possibly some pulling, transfer when pressed and/or rubbed, and sometimes a difficult removal.

Other suggestions exist for obtaining a glossy, non-transfer deposit, but generally come in the form of products to be applied in 2 steps (products called ‘double-step’ products): a first colored layer in contact with the lips, long-lasting, non-transfer, but not glossy, and a 2^{nd} ultra-glossy transparent layer applied on the first (‘top coat’) which provides glossiness and non-stickiness, and which can transfer without leaving any trace of color.

Likewise for the eyes, there are no non-transfer and non-sticky glossy eyeshadows, nor non-transfer glossy liners or mascaras.

Therefore, new compositions for the care and/or makeup of keratin materials (including skin, lips, eyelashes), in a single step, with very good glossiness properties and improved non-transfer properties are sought. And, advantageously care and/or makeup compositions not containing silicone compounds (compositions called ‘silicone-free’ compositions), with very good glossiness properties and improved non-transfer properties are sought.

The Applicant tested several film-forming polymers and selected the best candidates, taking into account their film drying speed and their compatibility with a non-aqueous polar volatile solvent. The Applicant thus identified a specific combination of ingredients consisting of at least one polyurethane-type polymer and one alkylcellulose-type polymer, in an anhydrous composition comprising at least one non-aqueous volatile polar solvent and optionally in addition one or more non-volatile hydrocarbon oils, allowing to obtain an ultra-glossy film with very long wear and non-transfer properties.

The composition of the invention has excellent glossiness properties and a non-transfer result that is unprecedented for this type of glossiness composition, even with high levels of non-volatile oils. The composition of the invention also allows for easy removal.

### DISCLOSURE OF THE INVENTION

The present invention therefore relates to an anhydrous cosmetic composition comprising, in a physiologically acceptable medium:
(i) at least one alkylcellulose, preferably ethylcellulose,
(ii) at least one film-forming polyurethane polymer, and
(iii) at least one non-aqueous polar volatile solvent selected from among C1-C5 lower monoalcohols, volatile hydrocarbon oils selected from within the group consisting of optionally hydroxylated esters, ethers, dialkoxymethane, carbonates, and mixtures thereof.

Another object relates to a cosmetic method for caring for and/or making up keratin materials, in particular the skin and/or the lips, comprising the application to said keratin materials of a composition as defined according to the invention.

“Cosmetic composition” means any composition for cosmetic purposes, that is to say aesthetic purposes, which may come into contact with the superficial parts of the human body and more particularly with keratin materials, in particular human skin and/or lips.

“Physiologically acceptable medium” means any excipient suitable for topical use, in contact with keratin materials, without risk of toxicity, incompatibility, instability and/or allergic response.

“Keratin materials” according to the invention, means the skin and/or its appendages, in particular the skin, eyelashes, eyebrows, lips, and more particularly human skin and/or lips.

“Anhydrous” means in particular that water is preferably not deliberately added to the compositions but may be present in trace amounts in the various compounds used in the compositions. In particular, the composition according to the invention comprises less than 4% by weight of water, preferably less than 3%, preferably less than 2%, more preferably less than 1%, even more preferably less than 0.5% by weight of water, relative to the total weight of said composition, or is even completely free of water.

### DETAILED DESCRIPTION OF THE INVENTION

A first object therefore concerns an anhydrous cosmetic composition comprising, in a physiologically acceptable medium:
(i) at least one alkylcellulose, preferably ethylcellulose,
(ii) at least one film-forming polyurethane polymer, and
(iii) at least one non-aqueous polar volatile solvent selected from among C1-C5 lower monoalcohols, volatile hydrocarbon oils selected from within the group consisting of optionally hydroxylated esters, ethers, dialkoxymethane, carbonates, and mixtures thereof.

### Alkylcellulose

The composition of the invention therefore comprises at least one alkylcellulose. Among the alkylcelluloses, mention may be made of methylcelluloses and ethylcelluloses, and mixtures thereof, preferably ethylcelluloses.

According to one embodiment, the composition comprises ethylcellulose as alkylcellulose.

The remainder of the description will refer to ‘ethylcellulose film-forming polymer’, ‘ethylcellulose polymer’ or ‘ethylcellulose’ interchangeably.

‘Film-forming polymer’ means a polymer capable of forming, on its own or in the presence of an auxiliary film-forming agent, a continuous and adherent film on a support, in particular on the skin and/or the lips.

‘Ethylcellulose polymer’ according to the invention means a cellulose derivative in which some of the hydroxyl groups on the repeating glucose units are converted to ethyl ether groups.

Ethylcellulose is an ethyl ether of cellulose, comprising a chain consisting of ß-anhydroglucose units bound together by acetal bonds. Each anhydroglucose unit has three hydroxyl groups which can be substituted by ethoxy groups. The number of ethyl ether groups can vary and is characterized by the percentage of ethoxylated substitution. The ethoxylated substitution (also called “ethyl ether content”) is generally from 10 to 55%. According to a particular embodiment, the ethylcellulose polymers used in a cosmetic composition according to the invention are preferably polymers having a degree of substitution in ethoxy groups, ranging from 2.5 to 2.6 per anhydroglucose unit, in other words comprising an ethoxy group content ranging from 44% to 55%. Reference may in particular be made to the ethylcellulose polymer described in Dow Chemical application WO2018057381.

Mention can in particular be made of the ethylcellulose polymers with the INCI name: ETHYLCELLULOSE, marketed under the following names:
ETHOCEL™ references manufactured by DUPONT and distributed by CHEMPOINT, comprising, for example, ETHOCEL™ Standard 4, ETHOCEL™ Standard 7, ETHOCEL™ Standard 7 FP Premium, ETHOCEL™ Standard 10, ETHOCEL™ Standard 20, ETHOCEL™ Standard 45, ETHOCEL™ Standard 100, ETHOCEL™ Standard 100 FP Premium, with an ethoxylated substitution content of 48.0 to 49.5%;
references AQUALON™ ETHYLCELLULOSE of type-K, type-N and type-T, preferably of type-N such as N7, N100, available from Ashland, Inc; and
references ANTARON™ ECO available from Ashland, Inc.

This alkylcellulose polymer may be used as is, or pre-solubilized or pre-dispersed in a non-aqueous polar solvent such as those described below, to facilitate its dispersion in the composition. This non-aqueous polar solvent may be volatile or non-volatile.

According to a first method, the alkylcellulose polymer is used as is (in powder form).

For a formulation in an anhydrous composition according to the invention, the alkylcellulose polymer may advantageously be mixed with at least one non-volatile polar hydrocarbon oil (iv) selected from liquid C10-C26 saturated, unsaturated, linear or branched fatty alcohols, and homogenized, before incorporation into the rest of the composition.

According to another embodiment, the alkylcellulose polymer is pre-solubilized or pre-dispersed in a non-aqueous polar volatile solvent (iii) selected from volatile hydrocarbon oils selected from within the group consisting of optionally hydroxylated esters, ethers, dialkoxymethane, carbonates, and mixtures thereof, before incorporation into the rest of the composition.

The content of alkylcellulose, in particular ethylcellulose, in the composition of the invention will generally range from 1 to 15% by weight, preferably from 1.5% to 10%, more preferably from 2 to 8% by weight of dry (active) matter relative to the total weight of the composition.

Preferably, a content greater than or equal to 2% by weight of alkylcellulose, preferably ethylcellulose, relative to the total weight of the composition will be used.

According to a particular embodiment, the alkylcellulose content, preferably ethylcellulose, will range from 2% to 5% by weight relative to the total weight of the composition.

In particular, the composition according to the invention comprises ethylcellulose in a content ranging from 1 to 15% by weight, preferably from 1.5% to 10%, more preferably from 2 to 8% by weight of (active) dry matter relative to the total weight of the composition.

### Film-Forming Polyurethane Polymer

The composition also comprises at least one film-forming polyurethane polymer.

The polyurethane can be prepared according to polycondensation methods well known to the person skilled in the art.

According to a particular embodiment, the polyurethane present in the composition according to the invention is capable of being obtained, or is obtained, by a multi-step method comprising:
i) the formation of a prepolymer resulting from the reaction of isophorone diisocyanate (IPDI) with a mixture of polytetrahydrofurans (also known as polybutylene glycols or polytetramethylene glycols), said polytetrahydrofurans advantageously comprising an average number of 14 moles of butylene glycol for one and an average number of 28 moles of butylene glycol for the other), then
ii) reacting said prepolymer with 4,4′-methylenebis(cyclohexylamine) then reacting the residual isocyanate groups with ethanol.

According to a particular embodiment, the polyurethane polymer according to the invention is pre-solubilized in a non-aqueous polar solvent such as those described below, and in particular a non-aqueous polar volatile solvent selected from among C1-C5 lower monoalcohols, preferably ethanol.

According to a particular method, the product marketed under the name Baycusan® C 2000 with INCI name: Polyurethane-64, Ethanol, by the company Covestro will be used.

The molecular weight of the polyurethane polymer used according to the invention will generally range from 5000 to 100 000 Da, better still from 8000 to 60 000 Da.

The raw material containing the polyurethane polymer pre-solubilized in the non-aqueous polar solvent may be more or less concentrated; the person skilled in the art may thus adjust the amount of raw material to be used with regard to its polymer concentration and/or its molecular weight.

It may also be possible to use several polyurethane polymers of different molecular weight and/or different concentrations of polyurethane polymers to optimize the desired result in terms of film adhesion and non-transfer.

Baycusan® C 2000 is already known in ethanol-based sunscreens and hair styling products, but has never been used until now, to the Applicant's knowledge, in skin and/or lip care and/or makeup compositions, probably due to its high ethanol content which may cause discomfort and/or tightness on the skin and lips.

The total content of film-forming polyurethane polymer(s) in the composition of the invention will generally range from 5% to 35% by weight, preferably from 8% to 30% by weight, or even from 10% to 25% by weight of active or dry material (polyurethane) relative to the total weight of the composition.

According to a particular embodiment, the total content of film-forming polyurethane polymer(s) will be greater than or equal to 10% by weight of active or dry matter (of polyurethane) relative to the total weight of the composition. Preferably, the total content of film-forming polyurethane polymer(s) will range from 10 to 25% by weight, in particular from 10 to 20% by weight of active or dry matter (of polyurethane) relative to the total weight of the composition.

Thus, according to a particular and preferred embodiment, the anhydrous composition of the invention comprises:
(i) at least one alkylcellulose, preferably ethylcellulose, in a total content ranging from 1 to 15% by weight, preferably from 2% to 5% by weight relative to the total weight of the composition, and
(ii) at least one polyurethane polymer, in a total content ranging from 5% to 35%, preferably 10% to 25% by weight of active or dry material (of polymer) relative to the total weight of the composition.

More preferably, the anhydrous composition of the invention comprises:
(i) ethylcellulose, in a total content ranging from 1 to 15% by weight, preferably from 2% to 5% by weight relative to the total weight of the composition, and
(ii) at least one polyurethane polymer, in a total content ranging from 5% to 35%, preferably 10% to 25% by weight of active or dry material (of polymer) relative to the total weight of the composition.

The polyurethane polymer will advantageously be pre-solubilized or pre-dispersed in a non-aqueous polar solvent to facilitate its dispersion in the composition, preferably a non-aqueous polar volatile solvent for rapid drying of the film after application.

According to a particular embodiment, a polyurethane polymer pre-solubilized in a non-aqueous polar volatile solvent (iii) such as those described below and preferably selected from C1-C5 lower monoalcohols, preferably ethanol, in particular a polyurethane polymer with INCI name: Polyurethane-64 or INCI name: Polyurethane-64, Ethanol, such as that marketed under the name Baycusan® C 2000 by the company Covestro, will be used.

The ethylcellulose polymer may advantageously be pre-solubilized or pre-dispersed in a non-aqueous polar volatile solvent (iii) such as those described below and preferably volatile hydrocarbon oils selected from within the group consisting of optionally hydroxylated esters, ethers, dialkoxymethane, carbonates, and mixtures thereof.

According to another embodiment, the ethylcellulose polymer may be pre-solubilized or pre-dispersed in a non-volatile polar hydrocarbon oil (iv) as described below and preferably selected from liquid C10-C26 saturated, unsaturated, linear or branched fatty alcohols, preferably octyldodecanol.

### Non-Aqueous Volatile Polar Solvent

The composition will thus comprise (iii) at least one non-aqueous polar volatile solvent. The non-aqueous polar volatile solvent(s) will advantageously serve to pre-solubilize and/or pre-disperse at least one of the film-forming polymers described above, or even both of the polymers described above, in order to facilitate its (their) incorporation into the composition, and will also participate in the establishment and adhesion of the film to the keratin materials after application and evaporation.

According to a particular embodiment, at least the polyurethane polymer is pre-solubilized or pre-dispersed in a non-aqueous polar volatile solvent (iii) as defined below.

‘Volatile solvent or oil’, according to the invention, means a liquid having lost more than 20% by mass of its mass at 60 minutes, according to the protocol below. Volatile solvents participate in the establishment of the film on the keratin materials during application, and allow, after evaporation, the film to adhere to the keratin materials, with a sensation of bare skin, without material effect.

METHODOLOGY: 20 mg of the liquid (solvent) to be studied is weighed onto a 5 cm×5 cm PMMA plate using a micropipette and a precision balance. This liquid is spread with a finger over the entire plate. Then, the plate is placed in a ventilated chamber thermostatically controlled at 33 °C and 50% humidity. For each liquid, the test is carried out 3 times. The mass loss during drying is measured after 60 minutes. The mass lost is expressed according to the following calculation:

$$\% lost mass = \frac{{m}_{t 0} - {m}_{tx}}{{m}_{t 0}} \times 100 \qquad [ Math 1 ]$$

With mₜₓ corresponding to the remaining mass at the measured time (t60 min) and mₜ₀ corresponding to the mass applied initially.

the term “non-volatile solvent or liquid” means a solvent or liquid that does not meet the definition of volatile solvent described above.

‘Polar solvent’ means a solvent comprising at least one polar group. Polar groups are well known to the person skilled in the art; they may in particular be alcohol, ester, ether or carboxylic acid groups.

In particular, a polar solvent according to the invention may have a Polarity index value which is equal to or greater than a value of 4. The polarity index is a quantity calculated on the basis of thermodynamic quantities (solubility and change of state) which highlights the more or less polar character of a molecule.

For the polarity indices of solvents, reference will be made to the article by LR SNYDER: Classification of the solvent properties of common liquids; Journal of Chromatography, 92 (1974), 223-230.

Preferred polar solvents are those consisting of a compound comprising at least one polar covalent bond of the O—H type.

As a “non-aqueous polar volatile solvent” that can be used according to the invention, mention may in particular be made of C1-C5 lower monoalcohols, volatile hydrocarbon oils selected from within the group consisting of optionally hydroxylated esters, ethers, dialkoxymethane, carbonates, and mixtures thereof.

According to a particular and preferred embodiment, the non-aqueous volatile polar solvent (iii) is selected from lower C1-C5 mono-alcohols, preferably ethanol.

Thus, according to a particular and preferred embodiment, the polyurethane polymer is (pre) dispersed or (pre) solubilized in a non-aqueous volatile polar solvent selected from C1-C5 lower monoalcohols, preferably ethanol.

According to another embodiment, the non-aqueous volatile polar solvent (iii) is a volatile hydrocarbon oil selected from:
optionally hydroxylated esters, preferably comprising between 5 and 12 carbon atoms in total, preferably monoesters, and in particular C2-C6 monoalcohol and C3-C6 monocarboxylic acid esters, optionally hydroxylated, comprising between 5 and 11 carbon atoms in total, for example such as isopropyl hexanoate or ethyl lactate;
ethers (in particular monoethers), having from 4 to 10 carbon atoms in total, for example such as dipentyl ether;
dialkoxymethanes having from 3 to 11 carbon atoms in total, for example such as dimethoxymethane, diethoxymethane, or dibutoxymethane. Such compounds are in particular marketed respectively under the names methylal, ethylal and butylal by the company Lambiotte;
carbonates having from 3 to 11 carbon atoms in total, for example such as 15 dimethyl carbonate, diethyl carbonate, dipropyl carbonate, or dipentyl carbonate;
and mixtures thereof.

Thus, according to a particular embodiment, the alkylcellulose polymer, in particular ethylcellulose, is (pre) dispersed or (pre) solubilized in a non-aqueous volatile polar solvent (iii) selected from volatile hydrocarbon oils selected from within the group consisting of optionally hydroxylated esters, ethers, dialkoxymethane, carbonates, and mixtures thereof.

The total content of non-aqueous volatile polar solvent(s) in the composition of the invention will range in particular from 8% to 65% by weight, preferably from 10% to 50% by weight, more preferably from 12% to 35% by weight relative to the total weight of the composition.

According to a particular embodiment, the non-aqueous polar volatile solvent content, in particular C1-C5 monoalcohol, preferably ethanol in the composition of the invention will range in particular from 8% to 65% by weight, preferably from 10% to 50% by weight, more preferably from 12% to 35% by weight relative to the total weight of the composition.

Other Additional Oils (Distinct from the Volatile Polar Solvents Described Above)

According to a particular embodiment, the composition of the invention comprises one or more additional hydrocarbon oils, volatile or non-volatile, polar or non-polar, distinct from the volatile polar solvents described above, in particular one or more additional hydrocarbon oils (iv) selected from liquid C10-C26 saturated, unsaturated, linear or branched fatty alcohols, ester oils, C8-C19 linear or branched alkanes, and mixtures thereof.

The additional polar and non-volatile oils can act as a polar non-volatile solvent for at least one of the two film-forming polymers described above, and furthermore provide comfort and hydration properties to the keratin materials to which the compositions of the invention are applied.

According to a particular embodiment, the ethylcellulose polymer according to the invention is pre-dispersed or pre-solubilized in non-volatile polar hydrocarbon oil as described below.

“Hydrocarbon oil” means an oil formed essentially, or even consisting of, carbon and hydrogen atoms and which may comprise at least one oxygen and/or nitrogen atom, and free of Si, F and P atoms.

### Non-Volatile Hydrocarbon Oil

According to a particular embodiment, the composition comprises at least one non-volatile polar hydrocarbon oil, in particular selected from within the group consisting of:
liquid C10-C26 saturated, unsaturated, linear or branched fatty alcohols;
ester oils;
and mixtures thereof.

Among the ester oils, mention can in particular be made of:
esters of fatty alcohols and C8-C18 fatty acids, in particular coco-caprylate caprate which is an ester of fatty alcohol of natural origin (for example coconut), caprylic acid and capric acid,
fatty acid esters, in particular with 4 to 22 carbon atoms,
synthetic esters such as oils of formula R₁COOR₂ in which R₁ represents the residue of a linear or branched fatty acid containing from 4 to 40 carbon atoms and R₂ represents a hydrocarbon chain, in particular a branched chain, containing from 4 to 40 carbon atoms provided that R₁+R₂ is ≥16, such as for example isononyl isononanoate, 2-ethylhexyl palmitate, octyldodecyl neopentanoate, 2-octyldodecyl stearate, isostearyl isostearate, 2-octyldodecyl benzoate, isopropyl myristate, isopropyl palmitate, butyl stearate, hexyl laurate, 2-ethylhexyl palmitate, 2-hexyl-decyl, 2-octyl-decyl palmitate, 2-octyldodecyl myristate, 2-diethyl-hexyl succinate;
linear fatty acid esters having a total carbon number ranging from 35 to 70;
hydroxylated esters, preferably having a total carbon number ranging from 35 to 70, such as polyglycerol-2 triisostearate, isostearyl lactate, octyldodecyl hydroxystearate, diisostearyl malate, glycerin stearate; diethylene glycol diisononanoate;
esters of aromatic acids and alcohols comprising 4 to 22 atoms;
esters of fatty alcohols or C24-C28 branched fatty acids;
polyesters resulting from the esterification of at least one triglyceride of hydroxylated carboxylic acid(s) by an aliphatic monocarboxylic acid and by an aliphatic dicarboxylic acid, optionally unsaturated,
   and mixtures thereof.

The person skilled in the art will be able to adjust the percentage of additional oil(s) according to the percentages of polymers used to obtain the desired performance.

According to a particular and preferred embodiment, the composition comprises at least one non-volatile polar hydrocarbon oil selected from liquid C10-C26 saturated, unsaturated, linear or branched fatty alcohols.

Indeed, according to a particular embodiment, the alkylcellulose polymer, preferably ethylcellulose, is pre-dispersed or pre-solubilized in a non-volatile polar hydrocarbon oil (iv) selected from liquid C10-C26 saturated, unsaturated, linear or branched fatty alcohols, preferably octyldodecanol, before incorporation into the rest of the composition.

Advantageously, the liquid C10-C26 fatty alcohols are fatty alcohols, preferably branched when they comprise at least 16 carbon atoms.

Preferably, the liquid fatty alcohol comprises from 10 to 24 carbon atoms, and more preferably from 12 to 22 carbon atoms.

As particular examples of liquid fatty alcohols which can be used preferably in the composition of the invention, mention may in particular be made of lauryl alcohol, isostearyl alcohol, oleyl alcohol, 2-butyloctanol, 2-undecyl pentadecanol, 2-hexyldecyl alcohol, isocetyl alcohol, octyldodecanol and mixtures thereof.

According to an advantageous embodiment of the invention, the liquid C10-C26 fatty alcohol is selected from octyldodecanol.

Thus, according to a particular embodiment, the composition of the invention will comprise at least octyldodecanol.

The total content of non-volatile polar hydrocarbon oils in the composition of the invention will generally range from 5% to 75% by weight, preferably from 15% to 70% by weight relative to the total weight of the invention.

According to a particular embodiment, the composition comprises at least one liquid C10-C26 fatty alcohol, preferably octyldodecanol in a content ranging from 5% to 75% by weight, preferably from 15% to 70% by weight relative to the total weight of the composition.

The composition may further comprise one or more apolar, volatile or non-volatile hydrocarbon oils.

Examples of volatile non-polar hydrocarbon oils comprise C8-C19 linear or branched alkanes, in particular:
C8-C16 isoalkanes (also called isoparaffins) such as isododecane, isodecane, isohexadecane, and for example oils sold under the trade names ISOPAR® or PERMETYL®,
linear alkanes having hydrocarbon chains in:
   C9-C17, C10-C14, C11-C13 such as a mixture of undecane and tridecane, marketed by BASF Care Creations under the name Cetiol® Ultimate,
   C9-C12, C12-C14, such as those marketed by BIOSYNTHIS under the name VEGELIGHT® SILK (INCI name C9-12 ALKANE), VEGELIGHT® 1214LC,
   n-dodecane (C12) and n-tetradecane (C14), in particular sold by Sasol under the references PARAFOL® 12-97 and PARAFOL® 14-97 respectively, and
   C15-C19, such as those marketed by Seppic under the name EMOGREEN® L15,
   and mixtures thereof.

The total content of volatile apolar hydrocarbon oils in the composition of the invention, when present, will generally range from 3% to 20% by weight, preferably from 5% to 15% by weight relative to the total weight of the invention.

According to a particular and preferred embodiment, the composition of the invention comprises:
(i) at least one alkylcellulose, preferably ethylcellulose,
(ii) at least one film-forming polyurethane polymer, in particular a polyurethane polymer with INCI name polyurethane-64
(iii) at least one non-aqueous polar volatile solvent selected from among C1-C5 lower monoalcohols, preferably ethanol, and
(iv) at least one non-volatile hydrocarbon oil selected from within the group consisting of liquid C10-C26 saturated, unsaturated, linear or branched fatty alcohols, preferably octyldodecanol.

According to a particular embodiment, the film-forming polyurethane polymer is pre-solubilized in a non-aqueous polar volatile solvent (iii) selected from C1-C5 lower monoalcohols, preferably ethanol, to facilitate its dispersion in the composition.

And preferably, the alkylcellulose polymer is mixed with (iv) at least one non-volatile hydrocarbon oil selected from within the group consisting of liquid C10-C26 saturated, unsaturated, linear or branched fatty alcohols, preferably octyldodecanol, before incorporation into the rest of the composition.

### Other Ingredients

The composition of the invention may further comprise at least one additional ingredient selected from additional agents promoting adhesion to keratin materials, antioxidants, perfumes, preservatives, cosmetic active ingredients, coloring materials, fillers, and mixtures thereof.

According to a particular embodiment, the composition of the invention further comprises an additional agent promoting adhesion to keratin materials, in particular selected from within the group consisting of sugar esters, preferably sucrose acetate isobutyrate (SAIB).

### Coloring Materials

According to a particular embodiment of the invention, in particular for makeup compositions, the composition comprises at least one coloring material.

For the purposes of the present invention, the term “coloring material” means a compound capable of producing a colored optical effect when formulated in sufficient quantity in a suitable cosmetic medium.

A coloring material may be selected from organic or inorganic coloring materials, materials with an optical effect such as mother-of-pearl, and mixtures thereof, soluble or insoluble in the oily phase of the invention.

According to a particular embodiment, the coloring material(s) are selected from pigments, pearlescent agents, fat-soluble coloring agents, and mixtures thereof.

“Pigments” means white or colored, mineral or organic particles, insoluble in an aqueous solution, intended to color and/or opacify the resulting deposit. Mention can be made of mineral pigments, organic pigments, and composite pigments (that is to say pigments based on mineral and/or organic materials).

Among “mineral pigments” mention can be made of titanium dioxide (rutile or anatase), possibly surface-treated; black, yellow, red and brown iron oxides; manganese violet; ultramarine blue; chromium oxide; hydrated chromium oxide and ferric blue.

For compositions intended for the lips, examples include titanium dioxide; black, yellow, red and brown iron oxides and manganese violet.

Examples of “organic pigments” include D & C red no 19; D & C red no 9; D & C Red no 22; D & C Red no 21; D & C Red no 28; D & C Yellow no 6; D & C orange no 4; D & C orange no 5; D & C Red no 27; D & C red no 13; D & C Red no 7; D & C Red no 6; D & C Yellow no 5; D & C Red no 36; D & C Red no 33; D & C orange no 10; D & C yellow no 6; D & C Red no 30; D & C red no 3; D & C Blue 1; carbon black and lakes.

In particular, the coloring material(s) are present in the composition in a content ranging from 2% to 20% by weight, preferably from 3% to 15% by weight relative to the total weight of the composition.

According to a particular method, the total coloring material content will range from 2 to 10% by weight relative to the weight of the composition.

According to an advantageous embodiment, the composition according to the invention is silicone-free, that is to say that it comprises less than 1% by weight in total of silicone(s), preferably less than 0.5% by weight, preferably less than 0.1% by weight, even better does not comprise any at all (0%).

The term “silicone” means any compound comprising in its structure one or more Si—O—Si bonds.

### Galenic

The composition of the invention is an anhydrous composition.

Preferably, it will be a liquid composition.

For the purposes of the present invention, “liquid” means a fluid composition which is capable of flowing under its own weight, at 20 °C and atmospheric pressure (760 mm Hg), as opposed to compositions called solid compositions.

In particular, it will be a composition for care and/or makeup of keratin materials, in particular a composition for the lips, a composition for the skin such as an eyeliner, an eyeshadow or a luminous foundation or a composition for the eyelashes such as a mascara, in particular a gloss or non-transfer glossy lipstick for the lips.

The different embodiments of the compositions according to the invention are described above.

According to a particular embodiment, the composition of the invention is a liquid composition for makeup of the skin or lips comprising:
(i) at least one alkylcellulose, preferably ethylcellulose,
(ii) at least one film-forming polyurethane polymer, in particular a polyurethane polymer with INCI name polyurethane-64
(iii) at least one non-aqueous polar volatile solvent selected from among C1-C5 lower monoalcohols, preferably ethanol,
(iv) at least one non-volatile hydrocarbon oil selected from within the group consisting of liquid C10-C26 saturated, unsaturated, linear or branched fatty alcohols, preferably octyldodecanol, and
(v) at least one coloring material.

According to a particular embodiment, the film-forming polyurethane polymer is pre-solubilized in a non-aqueous polar volatile solvent (iii) selected from C1-C5 lower monoalcohols, preferably ethanol, to facilitate its dispersion in the composition.

And preferably, the alkylcellulose polymer is mixed with (iv) at least one non-volatile hydrocarbon oil selected from within the group consisting of liquid C10-C26 saturated, unsaturated, linear or branched fatty alcohols, preferably octyldodecanol, before incorporation into the rest of the composition.

### Method for Preparing the Composition

A person skilled in the art will adapt the method for preparing the composition according to the film-forming polymers used, pre-solubilized or pre-dispersed or not in suitable solvents (iii) or (iv) such as those described above.

According to a particular embodiment, a polyurethane polymer pre-solubilized in a non-aqueous polar volatile solvent (iii) selected from C1-C5 lower monoalcohols, preferably ethanol, is used to facilitate its dispersion in the composition.

One such polyurethane polymer is polyurethane-64 from Covestro, with the INCI name: polyurethane-64, ethanol.

According to a first embodiment, the composition of the invention is prepared according to the following method:
1) the ethylcellulose polymer and the non-volatile polar oil (for example octyldodecanol) are hot mixed (for example 95 °C) until homogenized (for example duration 1 hour and rotation speed of 700 rpm);
2) the pigments, when present, are ground in a non-volatile polar oil (for example octyldodecanol) and the pigment paste is then added and homogenized until the mixture cools to room temperature;
3) the polyurethane-64 polymer, pre-solubilized in ethanol, is then added at room temperature (to avoid evaporation of the alcohol, whose flash point is very low: 13 °C) and everything is homogenized; and
4) the final composition is packaged in a sealed container to prevent the alcohol from evaporating.

According to another embodiment, the composition of the invention is prepared according to the following method:
1) the ethylcellulose polymer is pre-solubilized or pre-dispersed in a non-aqueous polar volatile solvent (iii) selected from volatile hydrocarbon oils selected from within the group consisting of optionally hydroxylated esters, ethers, dialkoxymethane, carbonates, and mixtures thereof, until homogenization;
2) the pigments, when present, are ground in a non-volatile polar oil (for example octyldodecanol) and the pigment paste is then added and homogenized until the mixture cools to room temperature;
3) the polyurethane-64 polymer, pre-solubilized in ethanol, is then added at room temperature (to avoid evaporation of the alcohol, whose flash point is very low: 13 °C) and everything is homogenized; and
4) the final composition is packaged in a sealed container to prevent the alcohol from evaporating.

The person skilled in the art will adapt the duration, temperature and rotation speed depending on the oil (iii) selected.

### Cosmetic Method

The invention also relates to a cosmetic method for caring for and/or making up keratin materials, in particular the skin and/or the lips, comprising the application to said keratin materials of a composition as defined above.

Applied to the skin or lips, the compositions of the invention form a glossy, non-transfer film.

The present invention will now be illustrated in the following non-limiting examples. The % are expressed by weight of raw material relative to the total weight of the composition, unless otherwise indicated.

### EXAMPLES

### Example 1: Synergistic Effect Between Ethylcellulose and Polyurethane Polymer

Several lipstick compositions were prepared and evaluated according to the criteria of glossiness, non-transfer, film adhesion/hold and removal.

TABLE 1

| | % | % | % | % | % | | | |
|---|---|---|---|---|---|---|---|---|
| | Compar- | Compar- | Compar- | Compar- | Compa- | | | |
| | ative | ative | ative | ative | rative | | | |
| | Poly- | Poly- | Poly- | Ethyl- | Ethyl- | | | |
| | urethane- | urethane- | urethane- | cellulose- | cellulose- | % | % | % |
| Ingredients | free | free | free | free | free | Invention | Invention | Invention |
| ETHYL- | 30 | 30 | 30 | — | — | 2.5 | 3.5 | 5.0 |
| CELLULOSE | | | | | | | | |
| (ANTARONECO) | | | | | | | | |
| POLY- | — | — | — | 81 | 25 | 25 | 25 | 25 |
| URETHANE-64, | | | | (32.5% | (10% | (10% | (10% | (10% |
| ETHANOL | | | | polymer | polymer | polymer | polymer | polymer |
| (BAYCUSANC | | | | and | and | and | and | and |
| 2000) | | | | 48.5% | 15% | 15% | 15% | 15% |
| | | | | ethanol) | ethanol) | ethanol) | ethanol) | ethanol) |
| ETHANOL | 50 | Qsp100 | Qsp100 | — | — | — | — | — |
| OCTYL- | 30 | 30 | 25 | 17 | 73 | 70.5 | 69.5 | 68.0 |
| DODECANOL | | | | | | | | |
| (RISONOL | | | | | | | | |
| 20 SP MB) | | | | | | | | |
| PIGMENTS | 2 | 2 | 2 | 2 | 2 | 2 | 2 | 2 |

The compositions are prepared according to the following method:

In a beaker in a water bath (95 °C), the ethylcellulose polymer and octyldodecanol are mixed for 1 hour at a rotation speed of 700 rpm. The preparation is then de-bubbled by reducing the stirring.

Sucrose acetate isobutyrate is added and homogenized for 20 min at 500 rpm. The pigments are ground in octyldodecanol and the pigment paste is then added and homogenized until the mixture cools to room temperature.

The polyurethane-64 polymer is added at room temperature (to avoid evaporation of the alcohol, which has a very low flash point of 13 °C) and homogenized.

The final composition is packaged in a sealed container to prevent the alcohol from evaporating.

The properties of the compositions according to the invention and respectively comparative compositions (without ethylcellulose polymer or without polyurethane-64) are evaluated on a panel of experts as follows: the composition is applied to the hand and to the lips and different properties are evaluated:
Glossiness: the glossiness of the film is evaluated once dry; the ratings given are as follows:
+++: very glossy;
++: glossy;
Non-transfer: the non-transfer properties are evaluated by touching the film with a finger or kissing the hand (pressure non-transfer evaluation), or by rubbing the film with a finger or rubbing the lips on the hand (friction non-transfer evaluation); the ratings given are as follows:
+++: very good non-transfer with pressure and friction;
++: good non-transfer with pressure and friction;
+: non-transfer with pressure but less resistance to friction;
−: transfer with pressure and friction;
Film adhesion/hold: Film adhesion/hold is evaluated by wearing the product on the lips for at least 4 hours; the ratings given are as follows:
+++: very good adhesion of the film/hold;
++: good film adhesion/hold;
−: no film adhesion/hold; the film is peelable or comes off.
Removal: Removal is evaluated by removing makeup with cotton by wiping the lips (for the test with ethylcellulose alone, which is very difficult to remove, a two-phase makeup remover is necessary to facilitate removal). The ratings are as follows:
+++: very easy removal
−: is not removed.

The results are presented in the following table:

TABLE 2

| | Compar- | Compar- | Compar- | | | | | |
|---|---|---|---|---|---|---|---|---|
| | ison | ison | ison | Compar- | Compar- | Invent- | Invent- | Invent- |
| | (with | (with | (with | ison | ison | ion | ion | ion |
| | 30% | 30% | 30% | without | without | (2.5% | (3.5% | (5% |
| | ethyl- | ethyl- | ethyl- | ethyl- | ethyl- | ethyl- | ethyl- | ethyl- |
| | cellulose | cellulose | cellulose | cellulose | cellulose | cellulose | cellulose | cellulose |
| | without | without | without | (with 32.5% | (with 10% | and 10% | and 10% | and 10% |
| | poly- | poly- | poly- | poly- | poly- | poly- | poly- | poly- |
| Properties | urethane) | urethane) | urethane) | urethane) | urethane) | urethane) | urethane) | urethane) |
| Glossi- | ++ | ++ | ++ | +++ | +++ | +++ | +++ | +++ |
| ness | | | | | | | | |
| Non- | +++ | +++ | +++ | ++ | − | + | + | + |
| transfer | | | | Dry, | Greasy | non- | non- | non- |
| | | | | non-greasy, | film | transfer | transfer | transfer |
| | | | | peelable | that | with | with | with |
| | | | | film | transfers | pressure | pressure | pressure |
| Film | +++ | +++ | +++ | − | − | ++ | ++ | ++ |
| adhesion/ | | | | | | | | |
| hold | | | | | | | | |
| Removal | − | − | − | +++ | +++ | +++ | +++ | +++ |

These results show that ethylcellulose alone provides a glossy, non-transfer film but which is very difficult to remove; polyurethane alone provides a glossy film and total non-transfer properties (with a volatile solvent) but poor film adhesion/hold, with a film which peels off easily.

On the other hand, the combination of ethylcellulose and polyurethane in the compositions of the invention allows to have improved film adhesion properties, while retaining good pressure-resistance properties and good gloss properties, with easy removal. And this result is particularly surprising with such high non-volatile oil contents (here, octyldodecanol at contents ranging from 68 to 70% in the examples illustrated above).

### Example 2: Improvement of Non-Transfer Properties to Friction

The Applicant prepared and tested several compositions according to the invention with different products with INCI names: ethylcellulose, different polyurethane polymer contents and with or without the addition of an additional agent promoting adhesion, such as sucrose acetate isobutyrate (SAIB).

The compositions and evaluations of gloss and non-transfer, according to the protocols described in the previous example, are presented in the following table:

TABLE 3

| Ingredients | % | % | % | % |
|---|---|---|---|---|
| Ethylcellulose | 2.5 | 2.5 | | 2.5 |
| (ANTARON ECO) | | | | |
| Ethylcellulose | | | 5 | |
| (ETHOCEL STD 7) | | | | |
| Polyurethane-64 | 25 (10% | 25 (10% | 25 (10% | 35 (14% |
| (BAYCUSAN C2000) | polymer and | polymer and | polymer and | polymer and |
| | 15% ethanol) | 15% ethanol) | 15% ethanol) | 21% ethanol) |
| OCTYLDODECANOL | 66.5 | 61.5 | 64.0 | 51.5 |
| SUCROSE ACETATE | — | 5 | — | 5 |
| ISOBUTYRATE | | | | |
| PIGMENTS | 6 | 6 | 6 | 6 |

EVALUATIONS according to the protocols
and ratings described in example 1

| | | | | |
|---|---|---|---|---|
| Glossiness | +++ | +++ | +++ | +++ |
| Non-transfer | + | + | + | ++ |
| | non-transfer | non-transfer | non-transfer | non-transfer |
| | with | with | with | with |
| | pressure but | pressure but | pressure but | pressure and |
| | less good | less good | less good | good |
| | resistance | resistance | resistance | resistance |
| | to friction | to friction | to friction | to friction |

These results show that increasing the polyurethane content further improves the non-transfer properties, particularly non-transfer with friction.

The person skilled in the art will therefore be able to adjust the contents of polyurethane, ethylcellulose, octyldodecanol and, if necessary, add an additional adhesion agent to optimize the adhesion properties of the film and non-transfer.

The combination of an ethylcellulose polymer and a polyurethane-64 therefore allows to formulate glossy, non-transfer anhydrous compositions for keratin materials, particularly skin and lips.

This combination can therefore be used advantageously in skin makeup compositions (for example: glossy eyeliners and eyeshadows, ‘glow’ or luminous foundation) or lips (for example: glossy liquid lipsticks).

### Example 3: Examples of Formulations

### 3.1. Glossy, Non-Transfer Liquid Lipstick

TABLE 4

| Ingredients (INCI name | |
|---|---|
| and commercial reference) | % by weight |
| Ethylcellulose | 2.5 |
| (ANTARON ECO ETHYLCELLULOSE) | |
| Polyurethane-64 in | 25.0 (including 10% |
| ethanol (BAYCUSAN C2000) | polymer and 15% |
| | ethanol) |
| Iron oxide pigments | 6.0 |
| and red lakes | |
| Octyldodecanol | Qsp 100 |
| (RISONOL 20 SP MB) | |

The composition is prepared according to the protocol described in example 1. Applied to the lips, the composition forms a very glossy, non-sticky film that does not transfer when pressed.

### 3.2 Non-Transfer Glossy Lipstick

TABLE 5

| Ingredients (INCI name | |
|---|---|
| and commercial reference) | % by weight |
| Ethylcellulose (ANTARON | 2.5 |
| ECO ETHYLCELLULOSE) | |
| Polyurethane-64 in | 25.0 (including 10% |
| ethanol (BAYCUSAN C2000) | polymer and 15% |
| | ethanol) |
| Sucrose acetate | 5.0 |
| isobutyrate (SAIB 100) | |
| Glyceryl behenate/ | 3.0 |
| eicosadioate (Nomcort HK-G) | |
| Iron oxide pigments | 6.0 |
| and red lakes | |
| Octyldodecanol | Qsp 100 |
| (RISONOL 20 SP MB) | |

The composition is prepared according to the protocol described in example 1. Glyceryl behenate/eicosadioate is added as a stabilizing agent.

Applied to the lips, the composition forms a very glossy, non-sticky film that does not transfer when pressed.

### 3.3 Non-Transfer Glossy Liquid Eyeliner

TABLE 6

| Ingredients (INCI name | |
|---|---|
| and commercial reference) | % by weight |
| Ethylcellulose (ANTARON | 3.5 |
| ECO ETHYLCELLULOSE) | |
| Polyurethane-64 in | 20.0 (including 8% |
| ethanol (BAYCUSAN C2000) | polymer and 12% |
| | ethanol) |
| Sucrose acetate | 5.0 |
| isobutyrate (SAIB 100) | |
| Black iron oxide pigments | 10.0 |
| Octyldodecanol | Qsp 100 |
| (RISONOL 20 SP MB) | |

The composition is prepared according to the protocol described in example 1. Applied to the edge of the eyelids, a glossy, non-sticky line that does not transfer when pressed, is obtained.

### 3.4 Non-Transfer Liquid Eyeshadow

TABLE 7

| Ingredients (INCI name | |
|---|---|
| and commercial reference) | % by weight |
| Ethylcellulose (ANTARON | 3.0 |
| ECO ETHYLCELLULOSE) | |
| Polyurethane-64 in | 25.0 (including 10% |
| ethanol (BAYCUSAN C2000) | polymer and 15% |
| | ethanol) |
| Sucrose acetate | 5.0 |
| isobutyrate (SAIB 100) | |
| Red and brown iron | 6.0 |
| oxide pigments | |
| Octyldodecanol | Qsp 100 |
| (RISONOL 20 SP MB) | |

The composition is prepared according to the protocol described in example 1. Applied to the eyelids, the composition forms a very glossy, non-sticky film that does not transfer when pressed.

1. An anhydrous cosmetic composition comprising, in a physiologically acceptable medium:
(i) at least one alkylcellulose,
(ii) at least one film-forming polyurethane polymer, and
(iii) at least one non-aqueous polar volatile solvent selected from among C1-C5 lower monoalcohols, volatile hydrocarbon oils selected from within the group consisting of esters, ethers, dialkoxymethane, carbonates, and mixtures thereof.

2. The cosmetic composition according to claim 1, wherein it further comprises (iv) one or more additional hydrocarbon oils selected from liquid C10-C26 saturated, unsaturated, linear or branched fatty alcohols, ester oils, C8-C19 linear or branched alkanes, and mixtures thereof.

3. The cosmetic composition according to claim 1, wherein the alkylcellulose is present in the composition in a total content ranging from 1% to 15% by weight, relative to the total weight of the composition.

4. The composition according to claim 1, wherein the film-forming polyurethane polymer(s) is (are) present in the composition in a total dry matter content ranging from 5% to 35% by weight, relative to the total weight of the composition.

5. The composition according to claim 1, wherein the non-aqueous polar volatile solvent is selected from C1-C5 lower monoalcohols.

6. The composition according to claim 1, wherein the content of non-aqueous polar volatile solvent(s) in the composition ranges from 8% to 65% by weight, relative to the total weight of the composition.

7. The composition according to claim 2, wherein the additional hydrocarbon oil is a non-volatile polar oil selected from liquid C10-C26 saturated, unsaturated, linear or branched fatty alcohols, ester oils, and mixtures thereof.

8. The cosmetic composition according to claim 1, wherein it comprises:
(i) at least one alkylcellulose,
(ii) at least one film-forming polyurethane polymer,
(iii) at least one non-aqueous polar volatile solvent selected from among C1-C5 lower monoalcohols, and
(iv) at least one non-volatile hydrocarbon oil selected from within the group consisting of liquid C10-C26 saturated, unsaturated, linear or branched fatty alcohols.

9. The composition according to claim 1, wherein it further comprises an additional agent promoting adhesion to keratin materials.

10. The composition according to claim 1, wherein it further comprises a coloring material selected from pigments, pearlescent agents, fat-soluble coloring agents, and mixtures thereof.

11. The composition according to claim 1, wherein it comprises less than 1% by weight in total of silicone(s).

12. The composition according to claim 1, wherein the composition is a liquid composition.

13. The composition according to claim 1, wherein it is a composition for care and/or makeup of keratin materials.

14. A cosmetic method for caring for and/or making up keratin materials comprising the application to said keratin materials of a composition as defined in claim 13.

15. The composition according to claim 8, wherein it comprises:
(i) ethylcellulose,
(ii) a polyurethane polymer with INCI name polyurethane-64,
(iii) ethanol, and
(iv) octyldodecanol.
